(19) United States
(12) Patent Application Publication
(10) Pub. No.: US 2010/0055664 A1
(43) Pub. Date: Mar. 4, 2010

(54) ENCLOSED MEMBRANE-CLAMPING DEVICES FOR RUNNING BIOLOGICAL ASSAYS ON MEMBRANE SURFACES

(76) Inventors: FUYING ZHENG, EL CERRITO, CA (US); YIQI LUO, EL CERRITO, CA (US)

Correspondence Address: YIQI LUO, 10810 SAN PABLO AVE, APT A204, EL CERRITO, CA, 94530, US

(21) Appl. No.: 12/200,410

(22) Filed: Aug. 28, 2008

(51) Int. Cl. C12Q 1/00; C12M 1/00

(52) U.S. Cl. 435/4; 435/287.1

(57) ABSTRACT

An enclosed membrane-clamping (EMC) device is disclosed for running biological assays on membrane surfaces, such as Western blotting. The EMC device comprises a cover plate and a support plate, which can be coupled through a sealing mechanism. The cover plate, the support plate and the sealing mechanism are shaped such that their inner surfaces form one or more enclosed chambers. When in use, a blotting membrane is placed between the cover plate and the support plate, and clamped in the chamber formed in the EMC device. The EMC device is coupled with an assisting device to realize automation of manipulations. Liquid-phase target solutions are introduced into the chamber through its inlet and outlet to realize surface-molecular interactions between the target on the blotting membrane and the targets introduced in liquid phase. The chamber with a small vertical dimension is capable of achieving the blotting assays at a higher speed.

### TECHNICAL FIELD

This invention relates generally to devices for running biological assays on membrane surfaces and methods for running biological assays on membrane surfaces using an enclosed membrane-clamping device.

### BACKGROUND

In the field of biological research, blotting assays such as Western blotting, Northern blotting, Southern blotting and dot blotting, etc. are considered some of the most powerful techniques for the identification and quantification of biological samples. In these assays, surface-molecular interactions, be it immunopair interactions or nucleic acid hybridizations, are most frequently carried out on a blotting membrane. The blotting membrane serves as a platform that carries some immobilized target on its surface. When each additional target dissolved in liquid is brought into contact with the membrane surface, the additional target is successively bound to the immobilized target through surface-molecular interactions between the different targets. Finally, the targets collected on the blotting membrane, for example, complexes of biological molecules, are detected and measured to achieve the purpose of the assays.

Traditionally, the surface-molecular interactions in these assays are carried out in open containers with a capacity from submilliliters to milliliters. In all these assays, the procedure of carrying out surface-molecular interactions follows similar protocols. In most cases, a blotting membrane is first stained with a sample containing the target to be measured. The staining may be implemented through various methods, such as the electrotransfer used in a Western blot. Additional targets are dissolved in liquids, and the stained blotting membrane is placed and submerged into successive containers holding the resulting solutions for the appropriate surface-molecular interactions to take place. Usually, the containers are shaken to increase the rate of surface-molecular interactions.

As a more specific example, in a Western blotting, before carrying out the surface-molecular interactions, the blotting membrane is stained with a sample, usually an antigen; then a primary antibody against the antigen and a secondary antibody against the primary antibody are added to form a complex with the antigen on the surface of the blotting membrane; then the secondary antibody is conjugated with a reporter such as a fluorophore or an enzyme; and last the signal from the fluorophore or enzyme is measured to quantify the staining sample on the blotting membrane.

There are two major limitations with the traditional method of carrying out the surface-molecular interactions in these assays. First, shaking of the containers has to be mild to prevent liquids from spilling, and that results in relatively thick diffusion layers formed at the membrane-liquid interface. The thicker diffusion layers hamper the mass transport in the liquid phase and hence impede the rate of the surface-molecular interactions and the speed of the assays. Second, lots of tedious manual manipulations are involved in the traditional method, such as swirling the blotting membrane in target solutions or pipetting liquids.

The present invention seeks to address these limitations by using proper devices to carry out the surface-molecular interactions when running the assays.

### SUMMARY

Devices for running biological assays on membrane surfaces are disclosed. An enclosed membrane-clamping device includes: a cover plate; a support plate; a sealing mechanism, wherein the cover plate and the support plate each has an inner surface facing each other; and wherein the sealing mechanism is located on either the cover plate, the support plate, or both plates, or is a standalone part capable of attaching to both plates; and wherein the cover plate, the support plate and the sealing mechanism are shaped such that their inner surfaces form one or more enclosed chambers when the cover plate and the support plate are coupled together by the sealing mechanism; and wherein lateral dimensions of the inner surface area on the support plate within each of the one or more enclosed chambers are sufficient to accommodate a customarily sized blotting membrane for purposes of running biological assays; and wherein vertical dimension of each of the one or more enclosed chambers are in the range of 0.1 millimeter to 1 centimeter; an inlet for the inflow of a target fluid into each of the one or more enclosed chambers; and an outlet for the outflow of the target fluid from each of the one or more enclosed chambers.

An additional device utilizing the enclosed membrane-clamping device in running biological assays includes: a first support platform capable of carrying one or more containers for target fluids; a second support platform capable of carrying one or more pairs of inlet and outlet tubes each of which is coupled to an enclosed chamber of the enclosed membrane-clamping device via its pair of inlet and outlet, and wherein the first and the second platforms are capable of specific relative transverse and vertical movements such that the one or more pairs of inlet and outlet tubes carried by the second support platform can be submerged in and taken out of each of the one or more containers for target fluids carried by the first support platform; a pumping device capable of being coupled to the one or more enclosed chambers of the enclosed membrane-clamping device and driving the movement of target fluids into the inlets, through the one or more enclosed chambers, and out of the outlets of the enclosed membrane-clamping device.

A method of using the disclosed devices in running biological assays is also disclosed which includes: receiving an enclosed membrane-clamping device; placing a customarily-sized blotting membrane on the support plate and within the bounds of one of the enclosed chambers to be formed; coupling the cover plate and the support plate together with the sealing mechanism such that the blotting membrane is sealed inside one of the enclosed chambers formed; coupling the inlet and outlet of the enclosed chamber containing the blotting membrane to a reservoir of a first target via a pumping device; continually pumping the first target into the enclosed chamber through the inlet and out of the enclosed chamber through the outlet of said enclosed chamber until a desired condition is met.

### DESCRIPTION OF DRAWINGS

In order to better understand the present invention and appreciate its practical applications, the following figures are provided and referenced hereafter. It should be noted that the figures are given as examples and by no means limit the scope of the invention as defined in the appended claims.

FIG. 1 illustrates an example enclosed membrane-clamping (EMC) device and an assisting device which manipulates the EMC device to carry out a biological assay.

FIG. 2 illustrates a three-dimensional view of an example EMC device.

FIG. 3 shows the cross-sectional views of some possible variations in the formation of an enclosed chamber in the EMC device by coupling the cover plate and the support plate via a sealing mechanism. FIG. 3A shows the coupling of a plane cover plate and a hollowed support plate to form an enclosed chamber in which a blotting membrane is clamped on the support plate. FIG. 3B shows the coupling a hollowed cover plate and a plane support plate to form an enclosed chamber in which a blotting membrane is clamped on the support plate. FIG. 2C shows the coupling of a cover plate and a support plate by means of double-sided adhesive tapes. FIG. 2D shows the coupling of the cover plate and the support plate by means of mortise and tenon joints.

FIG. 4 shows some possible variations of a clamping feature on the cover plate of an EMC device for holding a blotting membrane to the inner surface of the support plate. FIG. 4A illustrates a clamping feature on a plane cover plate holding down a blotting membrane to a hollowed support plate on one or both ends. FIG. 4B illustrates a clamping feature on a hollowed cover plate holding down a blotting membrane to a plane support plate on one or both ends. The cross-sectional view of the EMC device is used.

FIG. 5 illustrates some possible variations in the shape of the enclosed chamber of an example EMC device, and in the location, distribution and number of the inlets and outlets. The top view of the EMC device is used. FIG. 5A shows an EMC device having an enclosed chamber with rectangular sides. FIG. 5B shows an EMC device having an enclosed chamber with streamlined sides. FIG. 5C shows guiding canals or grooves inside the enclosed chamber for even distribution of fluid flow across the enclosed chamber from the inlet to the outlet. FIG. 5D illustrates some possible locations of multiple inlets and outlets on an example EMC device. FIG. 5E illustrates some relevant dimensions of an EMC device with a rectangular chamber shape.

FIG. 6 illustrates some of the possible variations of the fluidic guiding patterns on the cover plate of an example EMC device. FIG. 6A shows the cross-sectional view of an example EMC device with fluidic guiding patterns on the inner surface of the cover plate. It also shows the dimensions and variations of the cross section of the fluidic guiding patterns. FIGS. 6B, 6C, 6D and 6E illustrate some of the ways of patterning the inner surface of the chamber in an EMC device. The top view of the EMC device is used.

FIG. 7 illustrates the assembly of multiple EMC devices or an EMC device with multiple enclosed chambers and corresponding assisting devices to carry out several biological assays in parallel.

FIG. 8 is a flow diagram of an example process for carrying out a blotting assay using an EMC device.

### DETAILED DESCRIPTION

### Principle and Implementation of Solutions

The first drawback of a traditional assay method is the thicker diffusion layers near the blotting membrane: they hamper the mass transport in the liquid phase and hence impede the rate of the surface-molecular interactions and the speed of the assays. Chambers with a small vertical dimension enhance the interaction between mobile molecules present in a fluid and immobilized molecules present on a solid surface because the miniaturization significantly increases the contact efficiency between the two forms of molecules. The advantages of miniaturization apply to the membrane-based blotting assays, in which the interactions between molecules are carried out on the surfaces of blotting membranes. For example, detection of targets on a blotting membrane in the post-transfer steps in a Western blotting assay may be significantly accelerated when the process occurs in a miniaturized device. The present invention discloses a device for blotting assays, which is capable of achieving blotting assays in a chamber at a higher speed.

The second drawback of a traditional assay method is the tedious manual manipulations, such as swirling the blotting membrane in target solutions or pipetting liquids. With appropriate assisting devices that are capable of controlled manipulation of the disclosed device, many of the manual manipulations can be automated.

The present disclosure discusses a number of possible implementations that address the aforementioned drawbacks and create desirable features in devices for applying blotting assays. To facilitate discussion, examples are set forth in the context of an enclosed membrane-clamping (EMC) device. However, the same principles apply to other devices and other experimental contexts. The examples should not be construed to limit the claims in these respects.

### Example Assembly of an EMC Device and an Assisting Device

In the field of biological research, blotting assays including Western blotting, Northern blotting, Southern blotting and dot blotting have been enlisted as the most powerful techniques for the identification and quantification of samples. While applying these assays, surface-molecular interactions, either immunopair interactions or nucleic acid hybridizations, are carried out on a blotting membrane. The traditional method of carrying out the surface-molecular interactions has limitations such as insufficient speed and involvement of a lot of manual manipulations.

In order to increase the efficiency of carrying out the surface-molecular interactions in the blotting assays, a variety of enclosed membrane-clamping (EMC) devices for running biological assays on blotting membranes, assisting devices for automating the manipulations of the EMC devices, and methods for running biological assays on the blotting membranes clamped in the EMC devices are disclosed in the present disclosure.

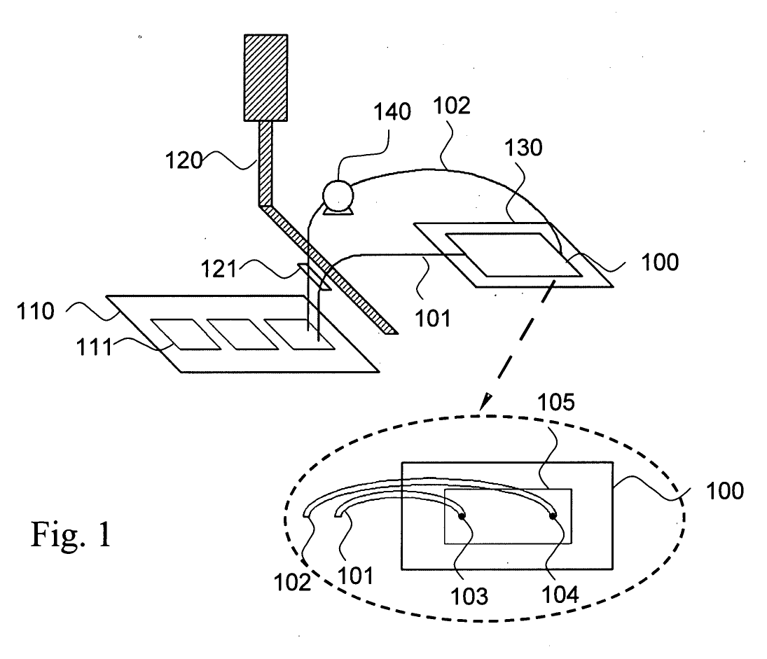

FIG. 1 illustrates an example enclosed membrane-clamping (EMC) device 100 and an assisting device which manipulates the EMC device to carry out a biological assay. The assisting device comprises a first support platform 110, a second support platform 120, a third support platform 130 and a pumping device 140.

The example EMC device 100 has an enclosed chamber 105 with an inlet 103 and an outlet 104. When using the EMC device for assays, the blotting membrane is sealed inside the enclosed chamber 105 and targets in liquid phase are injected into the inlet 103, flow across the enclosed membrane in the enclosed chamber 105, and out of the outlet 104. In some implementations, the example EMC device 100 can include inlet and outlet tubing 101 and 102 attached to the inlet 103 and outlet 104. In some implementations, the example EMC device 100 can also include additional numbers of inlets and outlets.

In some implementations, the EMC device can be assembled to an assisting device comprising a first support platform 110, a second support platform 120, an third support platform 130 and a pumping device 140. The assisting device can be in the form of one or more integrated devices or standalone components that the user puts together ad hoc. In some implementations, the third platform is optional.

The first support platform 110 is capable of carrying one or more containers 111 of liquid-phase target solutions. The second support platform 120 is capable of carrying one or more pairs of inlet and outlet tubes 101 and 102 that are connected to the one or more pairs of inlet and outlet of the chamber 105 in the EMC device 100. The first and the second platforms are capable of specific relative transverse and vertical movements such that the one or more pairs of inlet and outlet tubes 101 and 102 carried by the second support platform 120 can be submerged in and taken out of each of the containers 111 carried by the first support platform 110. In some implementations, the first support platform is stationary, while the second support platform is capable of motions in all three dimensions. In some implementations, the first platform is capable of motions in all three dimensions while the second support platform stays stationary. In some implementations, the first support platform is capable of transverse motions while the second support platform is capable of vertical motions, or vice versa.

In some implementations, the transverse or vertical movements of the first and second support platform 110 and 120 can be effectuated by motors, levers, manual force, or other mechanical, electrical, magnetic mechanisms.

In some implementations, the movements of the first and second support platform 110 and 120 can be controlled by a programmable unit that is capable of determining the amount and timing of the movements. In some implementations, the programmable unit operably coupled to the first and the second support platform can be a computer processor, a timer, or other mechanisms by which a user can specify desired conditions for platform movements.

The pumping device 140 is capable of being coupled to the chamber 105 in the EMC device 100 or the one or more pairs of inlet and outlet tubes 101 and 102 for the purpose of driving the movement of target solutions into the inlets 103, through the chamber 105, and out of the outlets 104 of the EMC device 100. In some implementations the pumping device can be connect to either the inlet tubing, or the outlet tubing or both. In some implementations, the pumping device 140 can be a peristaltic pump. In some implementations, the pumping device 140 is capable of reversing the direction of the fluid flow. In some implementations, the pumping device 140 is capable of being coupled to multiple chambers in an EMC device or multiple EMC devices at the same time.

In some implementations, the pumping device 140 can be controlled by a programmable unit that is capable of determining the direction and timing of the movements. In some implementations, the programmable unit operably coupled to the pumping device can be a computer processor, a timer, or other mechanisms by which a user can specify desired conditions for pump direction, timing and other relevant pumping parameters.

In some implementations, the third support platform 130 is employed for carrying the EMC device 100. The third support platform 130 is capable of orienting the EMC device such that the outlet 104 is higher than the inlet 103 of the enclosed chamber in the EMC device 100 when the first target or each of the successive target solutions is continually pumped into the chamber 105. The third support platform 130 is also capable of orienting the EMC device 100 such that the outlet 104 is lower than the inlet 103 when the first target and each of the successive target solutions is being emptied out of the chamber in preparation of the injection of the washing liquid or the next target solution. The movement of the third support platform can also be effectuated through manual force or other mechanisms as with the first and second support platforms. In some implementations, the third support platform is coupled to a programmable unit capable of controlling the movement of the third support platform on specific timing and conditions.

In some implementations, the pumping device 140 is capable of reversing pumping direction and the third support platform 130 does not need to move. In such implementations, the EMC device can be fixed in orientation and the outlet 104 is higher than the inlet 103 of the enclosed chamber 105 in the EMC device 100 when the first target or each of the successive targets is to be continually pumped into the chamber. When emptying the chamber in preparation of the injection of the next target, the pumping direction is reversed and the roles of the inlet and outlet are temporarily reversed and the liquid inside the chamber is emptied out via the inlet of the EMC device.

The purpose behind orienting the EMC device in a certain way or reversing the pumping direction as described above is to reduce the accumulation of bubbles in chamber of the EMC device. As target solutions travel through the enclosed chamber, bubbles in the solutions tend to be trapped inside the enclosed chamber, and hinder the flow of the target solutions. By placing the outlet higher than the inlet, bubbles inside the chamber have a better chance of escaping with the fluid flow. When a target solution is being emptied out of the chamber, air can better drive the liquid out when the liquid is emptied out of the lower opening of the chamber. The orientations of the EMC device or the pumping direction are example implementations of this principle, and they are not to be interpreted to limit the claims unless specified by the claim language.

In some implementations, a feature 121 is applied to affix the inlet and outlet tubes 101 and 102. One of the purposes of this feature is to maintain the positions of the inlet and outlet tubes and their separation distance. In most cases, the ends of inlet and outlet tubes are spaced close enough to keep them submerged in the same container of a liquid-phase target solution. At the same time, the distance between ends of the inlet and outlet tubes should be sufficiently large to ensure that fresh target solution is circulated in the chamber of the EMC device. In some implementations, the feature 121 is affixed to the second support platform 120. In some implementations, the feature 121 is a standalone feature. In some implementations, the feature 121 is affixed to a container on the first support platform 110 or the first support platform 110 itself.

### An Example EMC Device

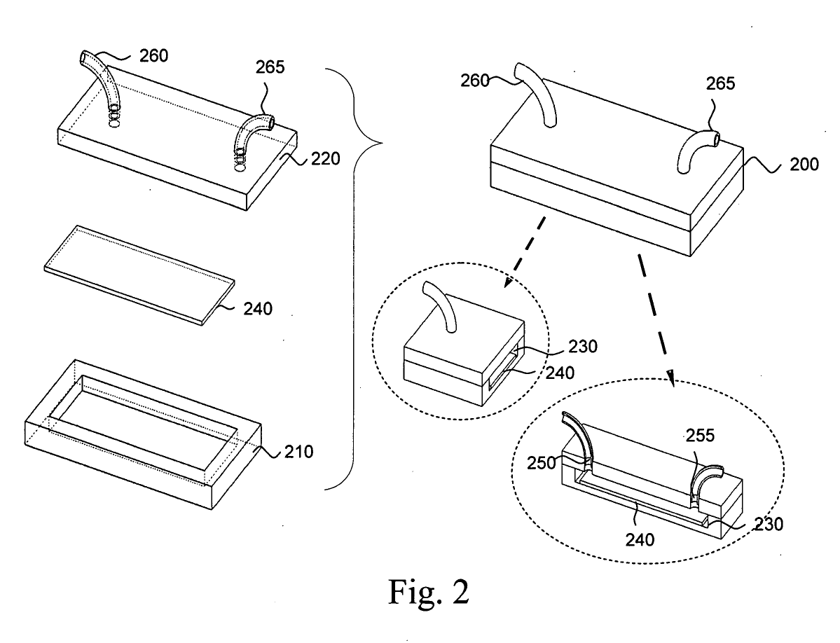

FIG. 2 illustrates a three-dimensional view of an example EMC device 200. On the left side of FIG. 2, the basic special relationship between different parts of an EMC device and a blotting membrane is shown. On the right side, a sealed EMC device with a blotting membrane enclosed in its chamber is shown. The lower right of FIG. 2 shows three dimensional cross-section of the EMC device along the longitudinal direction and the crosswise direction.

In some implementations, an EMC device 200 comprises a support plate 210 and a cover plate 220. The support plate 210 and the cover plate 210 can be coupled through a sealing mechanism (not shown in the figure). The inner surfaces of the support plate 210 and the cover plate 220 faces each other when the two plates are sealed together by the sealing mechanism. In some implementations, the support plate 210 has a hollowed region in the middle, and the inner surfaces of the cover plate 220 and the support plate 210 form an enclosed chamber 230 when sealed together. In some implementations, the cover plate 220 and the support plate 210 can be provided as separate parts. In some implementations, the cover plate 220 and the support plate 210 can be provided already attached to each other on one side. For example, the two plates can be attached along one edge with some flexible hinges for easy opening and closing.

When in use, a blotting membrane 240 is placed on the support plate 210 within the hollowed region such that when the two plates are coupled together, the reactive area of the blotting membrane or the whole membrane is within the enclosed chamber 230. In some implementations, a commercially available blotting membrane can be used. As an example, a commercially available blotting membrane is 8 cm×8 cm. Examples of the dimensions of the blotting membrane can be 8 cm×8 cm, 8 cm×4 cm, 8 cm×8/3 cm, 8 cm×2 cm, 8 cm×1 cm, 4 cm×4 cm, 4 cm×2 cm, or 4 cm×1 cm and so on. A customarily sized blotting membrane 240 can be any of the dimensions a user elects to use, but is generally those commonly used by practitioners in the field. The lateral dimensions of the enclosed chamber 230 formed by the cover plate 220 and the support plate 210 is sufficient to hold a customarily sized blotting membrane. For example, the lateral dimensions of surface on the support plate 210 within the enclosed chamber 230 can be slightly larger than 8 in×8 in, 8 in×4 in, 8 in×8/3 in, 8 in×2 in, 8 in×1 in, 4 in×4 in, 4 in×2 in, or 4 in×1 in and so on. When in use, the cover plate and the support plate are often interchangeable. The support plate supports the blotting membrane during the assays and is named as such. The support plate can be either on the top or the bottom of the EMC device when in use.

In some implementations, through holes can be made on either the cover plate 220 or the support plate 210 to serve as inlet 250 and outlet 255. In some implementations, the inlet 250 and outlet 255 are placed close to the two longitudinal ends of the enclosed chamber 230. In most cases, the inlet 250 and outlet 255 can be used interchangeably, and the labels are given according to the flow direction of target solutions during the assays. In some implementations, the inlet 250 and outlet 255 are capable of being coupled to inlet and outlet tubes 260 and 265. In some implementations, the inlet and outlet of the EMC device are integrated with inlet and outlet tubes.

In some implements, the inlet 250 and outlet 255 are connected to tubes 260 and 265 for the purpose of transporting liquid-phase target solutions into the chamber 230. The tubes may be inserted into a portion of the inlet 250 and outlet 255, or inserted through the inlet 250 and outlet 255. In some implementations, the coupling between the tubes and the inlet and outlet may be realized by friction or glue.

An example EMC device 200 is capable of accelerating the surface-molecular interactions in blotting assays compared to the traditional method. This advantage partly results from the shape of the enclosed chamber 230. The vertical dimension of the enclosed chamber is much smaller compared to the length and width of the enclosed chamber, so that the contact efficiency between the mobile target in the liquid phase and the immobilized target on the surface of the blotting membrane is enhanced.

The contact efficiency is defined as the collision rate between the two targets, which is a function of: (1) the concentrations of the two targets; and (2) the mass transport rate of the mobile target in the liquid phase towards the surface of the blotting membrane. The small height of the chamber shortens the average diffusion time of the mobile target within the liquid phase to the surface of the blotting membrane, resulting in an enhanced mass transport rate of the mobile target in the liquid phase. Moreover, at a typical flow rate of the liquid-phase target solutions, the flow velocity in the chamber is relatively high because the vertical dimension of the enclosed chamber restricts the cross-sectional area normal to the flow direction. A high flow velocity also enhances the mass transport rate of the target in the liquid phase.

The rate of the surface-molecular interactions is described as follows:

$$\begin{matrix}\frac{d [ AB ]}{d t} = {k}_{1} {\frac{[ B ]}{{[ B ]}_{0}} [ A ]}_{Surface} - {k}_{2} \frac{[ AB ]}{{[ B ]}_{0}} \\ = {k}_{1} \frac{[ B ]}{{[ B ]}_{0}} \frac{{{{k}_{m} [ A ]}_{Bulk} [ B ]}_{0} + {k}_{2} [ AB ]}{{{k}_{m} [ B ]}_{0} + {k}_{1} [ B ]} - {k}_{2} \frac{[ AB ]}{{[ B ]}_{0}} ;\end{matrix}$$

$$If {k}_{1} >> {k}_{m} and [ {B}_{0} ] \approx [ B ] ;$$

$$then \frac{d [ AB ]}{d t} \approx {{k}_{m} [ A ]}_{bulk} ;$$

$${k}_{m} ∝ {( \frac{v}{h} )}^{\frac{1}{3}} ;$$

where [A]_{Surface} and [A]_{Bulk} are the surface and bulk concentration of the mobile target in the liquid phase respectively, [B]₀ and [B] are the initial and real-time surface density of the immobilized target on the surface of the blotting membrane respectively, [AB] is the real-time surface density of the complex formed by the two targets, h is the height of the chamber, k₁ and k₂ are the forward and reverse rate constants of the surface-molecular interaction respectively, and kₘ is the mass-transport constant of the mobile target in the liquid phase (i.e., the diffusion coefficient divided by the thickness of diffusion layer). When k₁ is much larger than kₘ and [B]₀ and [B] are comparable, the interaction rate is proportional to [A]_{Bulk}, the concentration of the mobile target in the liquid phase. This is a mass-transport controlled rate, which is common in surface-molecular interactions. Because kₘ is positively correlated to the ratio of flow velocity v to height h, the rate of surface-molecular interactions should be enhanced by increasing flow velocity of the target solutions and decreasing the height of the chamber. In some implementations, the vertical dimension (or height) of the enclosed chamber is in the range of 0.1 mm and 1 cm. In some implementations, the vertical dimension (or height) of the enclosed chamber is in the range of 0.5 mm to 1 mm.

In some implementations, the cover plate, the support plate and the sealing mechanism can be made of one or multiple materials, each of the three can further be made of multiple materials. The materials can include polymers, plastics, glass, quartz, silicon, silicone, metals, and the like. Due to the mechanical strength and the ease of manufacturing, plastics may be a preferred choice for making the cover and support plates. Suitable plastics can include, but are not limited to, polycarbonate (PC), polystyrene (PS), polypropylene (PP), polyethylene (PE), polymethyl methacrylate (PMMA) and polyisoprene. The materials for the cover plate and the support plate should have sufficient hydrophilicity so that air bubbles are not easily formed when a liquid-phase target solution travels through the enclosed chamber. In some implementations, the inner surfaces of the cover plate and the support plate can be treated with another material (such as a special coating) to achieve better hydrophilicity. In some implementations, the materials for the cover plate and the support plate may be transparent such that the enclosed membrane is clearly visible from the outside to the naked eye or through a photographic device. In some implementations, the material for the cover plate and the support plate may be opaque.

In some implementations, the cover plate and the support plate are re-sealable, and thus making the EMC device reusable for different assays. In some implementations, the sealing mechanism can be sealed only once, and is not re-sealable or reusable once re-opened.

### Example Enclosed Chamber and Sealing Mechanism in an EMC Device

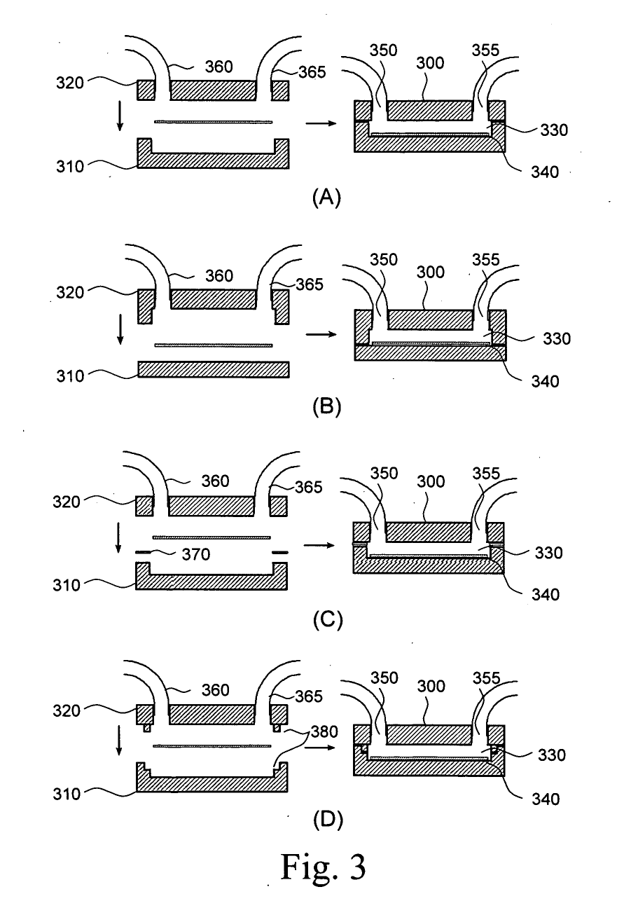

FIG. 3 shows the cross-sectional views of some possible variations in the formation of an enclosed chamber 330 in an example EMC device 300 by coupling the cover plate 320 and the support plate 310 via a sealing mechanism 370 or 380. On the left is the EMC device 300 before its parts are sealed together, and on the right is the sealed EMC device 300.

FIG. 3A shows the coupling of a plane cover plate 320 and a hollowed support plate 310 to form an enclosed chamber 330 in which a blotting membrane 340 is clamped on the support plate 310 within the enclosed chamber 330. The inlet 350 and the outlet 355 are bore in the cover plate 320, and connected to the inlet and outlet tubes 360 and 365.

FIG. 3B shows the coupling of a hollowed cover plate 320 and a plane support plate 310 to form an enclosed chamber 330 in which a blotting membrane 340 is clamped on the support plate 310 within the enclosed chamber 330. The inlet 350 and the outlet 355 are bore in the cover plate 320, and connected to the inlet and outlet tubes 360 and 365.

FIGS. 3A and 3B illustrates that the shapes of the cover plate 320 and the support plate 310 can vary, and the inner surface of each can be either flat or concave, or even convex, as long as their inner surfaces form an enclosed chamber when they are coupled together. In some implementations, the sealing mechanism can be part of either or both of the cover plate and the support plate, and in such cases, the sealing mechanism can be shaped such that the inner surfaces of the cover plate, the support plate and the sealing mechanism form an enclosed chamber when the cover plate and the support plate are coupled together via the sealing mechanism.

In some implementations, the surface of the support plate within the enclosed chamber is flat such that a blotting membrane can lay flat on the support plate and the fluid coming in from the inlet on the cover plate can push down and clamp it to the support plate without additional mechanism.

FIG. 3C shows the coupling of a cover plate 320 and a support plate 310 by means of double-sided adhesive tapes 370. The double-sided adhesive can be already attached on one side to one of the two plates, and has a protective covering on the other side. When a user is ready to seal the EMC device, the protective covering is pealed away, and the two plates can be brought together and pressed against each other for the double-sided tape to adhere to both plates. In some implementations, the adhesive tapes are provided separate from the plates, and needs to be adhered to both plates when sealing the EMC device. In some implementations, the sealing can be done with gluing.

FIG. 3D shows the coupling of the cover plate 320 and the support plate 310 by means of mortise and tenon joints 380. The cover plate 320 and the support plate 310 are shaped such that they creates the mortise and tenon, and can be fitted together tightly to provide a water-tight seal around the enclosed chamber 330.

FIGS. 3C and 3D illustrates that there can be many variations in the form of the sealing mechanism. The two plates each have an inner surface facing each other. The sealing mechanism can be located on either the cover plate, the support plate, or both plates, or is a standalone part capable of attaching to both plates. The enclosed chamber formed should be sufficiently water-tight such that when liquid is pumped through the enclosed chamber 330 during an assay, there would not be leakage of the liquid. In some implementations, other types of sealing mechanism, such as a mechanical seal can be used. In some implementations, additional parts such as rubber or plastic washers, O rings, and etc. can be used to improve the seal. In some implementations the plates can be already attached to each other on one side, and can be sealed on other sides when in use. The sealing mechanism can be resealable or non-resealable. In some implementations, the vertical dimension of the enclosed chamber is determined by the thickness of the sealing mechanism. In some implementations, the blotting membrane is clamped on the support plate by the sealing mechanism when the cover plate and the support plate are coupled together by the sealing mechanism.

### Clamping Feature of an Example EMC Device

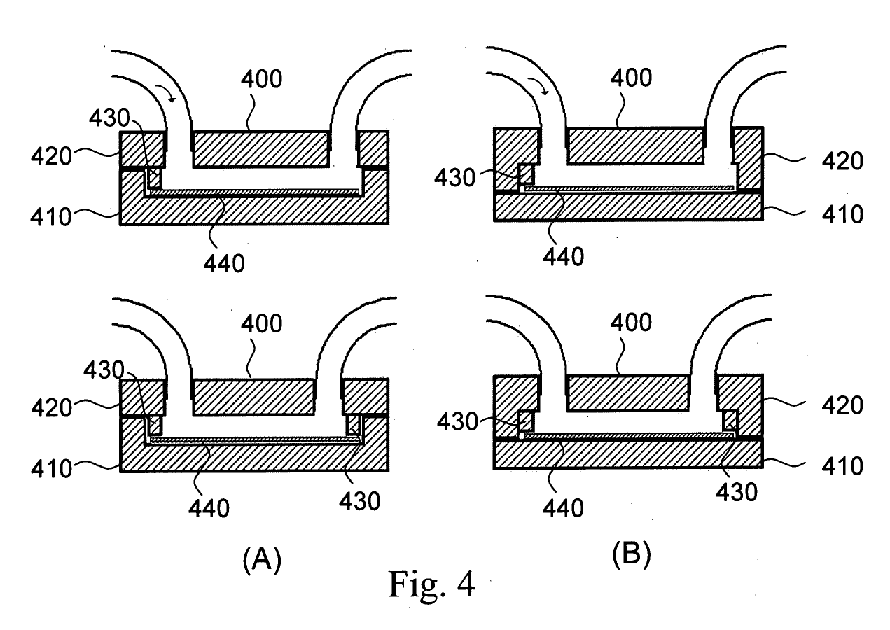

FIG. 4 shows some possible variations of a clamping feature on the cover plate of an EMC device 400 for holding a blotting membrane 440 to the inner surface of the support plate 410. FIG. 4A on the left illustrates a clamping feature 430 on a plane cover plate 420 holding down a blotting membrane 440 to a hollowed support plate 410 on one (top) or both ends (bottom). FIG. 4B on the right illustrates a clamping feature 430 on a hollowed cover plate 420 holding down a blotting membrane 440 to a plane support plate 410 on one (top) or both ends (bottom). The cross-sectional view of the EMC device along the longitudinal direction is used.

In some implementations, a clamping feature is added to the EMC device to fix the position of the blotting membrane so that it would not float around within the enclosed chamber of the EMC device during the assay. In some implementations, no special clamping feature is required because a wet blotting membrane automatically adheres to the surface of the support plate made of certain materials.

FIG. 4 illustrates that the addition of clamping features to the cover plate 420 such that one or both longitudinal ends of the customarily sized blotting membrane 440 can be clamped between the inner surfaces of the clamping features 430 and the support plate 410 when the EMC device 400 is sealed. In some implementations, if there is only one clamping feature on the cover plate, the opening closer to the clamping feature is often used as the inlet.

In some implementations, when the support plate has a hollowed region in the middle, the clamping features 430 may be protrusions close to the longitudinal ends of the cover plate 420, as shown in FIG. 4A. In some implementations, when the cover plate has a hollowed region in the middle, the clamping features 430 may be part of the edges of the cover plate 420, as shown in FIG. 4B. The clamping features can be made of the same material as the cover plate, or different material such as soft polymers for the purpose of tight clamping of the blotting membrane. In some implementations, the clamping feature may be provided separately and added to the chamber when the device is in use. An example of this kind of clamping feature can be an inert gel bead that can be placed on top of the blotting membrane at one or both ends and when the cover plate is coupled to the support plate, the gel bead will be squeezed and pushed down on the blotting membrane against the support plate.

### Example Enclosed Chamber and Sealing Mechanism in an EMC Device

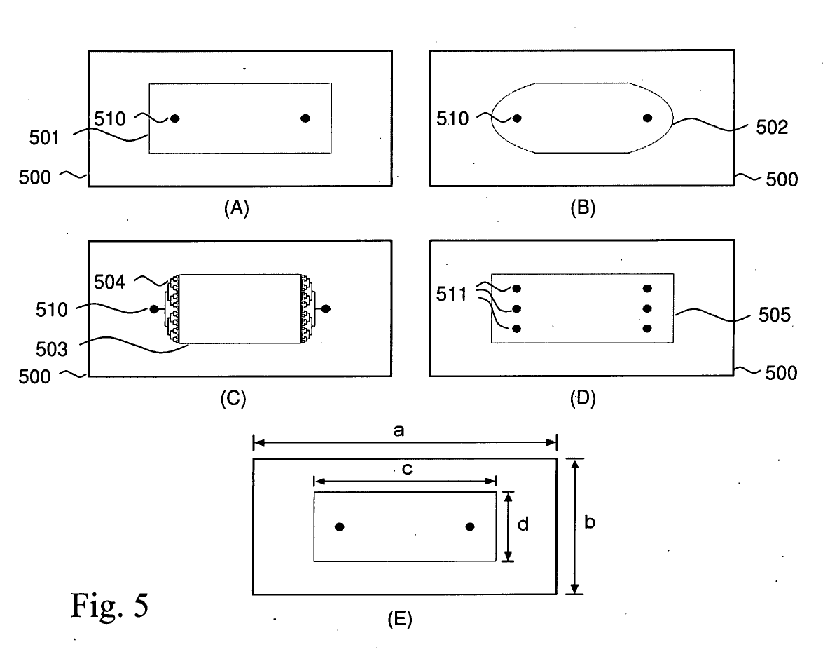

FIG. 5 illustrates some possible variations in the shape of the enclosed chamber of an example EMC device 500, and in the location, distribution and number of the inlets and outlets. The top view of the EMC device 500 is used. FIG. 5A shows an EMC device 500 having an enclosed chamber 501 with a rectangular shape. FIG. 5B shows an EMC device 500 having an enclosed chamber 502 with a streamlined shape. FIG. 5C shows guiding canals or grooves 504 inside the enclosed chamber 503 for even distribution of fluid flow across the enclosed chamber 503 from the inlet 510 to the outlet. FIG. 5D illustrates some possible locations of multiple inlets 511 and outlets in the enclosed chamber 505 of an example EMC device 500. FIG. 5E illustrates some relevant dimensions of an EMC device with a rectangular chamber shape.

FIGS. 5A and 5B show some variations of the shape of the chamber in an EMC device 500 in the lateral dimensions. FIG. 5A shows a rectangular chamber 501 and FIG. 5B shows a streamlined chamber (such as an elliptically-shaped chamber) 502. The inlet and outlet of the enclosed chambers are located close to the two longitudinal ends of the enclosed chambers. The inlet and outlet should keep a small distance from the ends for the ease of manufacturing and for the possibility of adding features at the ends. Compared to a rectangular shape, a streamlined shape eliminates the corners within the enclosed chamber so that the liquid-phase target solutions can be emptied from the chamber more easily. In some implementations, the enclosed chamber can be streamlined on all sides (3D) to facilitate the flow of the fluid inside.

FIG. 5C shows a rectangular chamber 503 connected to channel networks 504. In some implementations, the channel network 504 can made of canals or grooves on inner surface the cover plate. In some implementations, the channel networks can be a tree-like structure with branches that can evenly distribute the target solution from the inlet across the enclosed chamber. Similarly, a tree-like structure can also be used to collect the target fluid across the enclosed chamber at the outlet. This feature is particularly useful when the lateral dimension across the enclosed chamber is relatively large compared to the size of the inlet and outlet. With the tree-like structure near the inlet and the outlet, the uniformity of the flow field in the chamber may be enhanced.

FIG. 5D shows a rectangular chamber 505 with multiple pairs of inlet and outlet 520 in an example EMC device 500. The number and distribution pattern of inlets and outlets can influence the flow field in the enclosed chamber and should be adjusted according to the need of flow field. In some implementations, the number of inlets and outlets can be different. In some implementations, the positions and layout of the inlets and outlets are not limited to the longitudinal ends of the enclosed chamber and can be anywhere on the EMC device. When the lateral dimension across the enclosed chamber is relatively large, more inlets across the enclosed chamber may facilitate the uniformity of the flow field within the chamber. In some implementations, the multiple inlet and outlet tubes connected to the multiple inlets and multiple outlets can in turn be connected to a single inlet tube and a single outlet tube respectively. In some implementations, the tree-like tubing structure for the multiple inlets and multiple outlets can be provided separately, or already integrated to the inlets and outlets.

FIG. 5E shows the relevant dimensions of an example EMC device 500 having a rectangular chamber. The longitudinal dimension of the EMC plates is denoted as “a”, the crosswise dimension of the EMC plates is denoted as “b”, the longitudinal dimension of the enclosed chamber is denoted as “c”, and the crosswise dimension of the enclosed chamber is denoted as “d”.

In some implementations, the edge thicknesses “(a-c)/2” and “(b-d)/2” can be set in the range from one millimeter to a few centimeters. This edge thickness can be adjusted for realizing the sealing mechanism, ease of manipulation, and manufacturing.

The length “c” and the width “d” of the enclosed chamber should be comparable to the dimensions of a customarily sized blotting membrane and sufficient to accommodate the reactive area of the blotting membrane or the entire blotting membrane. In some implementations, the lateral dimensions “c” and “d” of the enclosed chamber can be ranged from a 5 millimeters to 10 centimeters.

### Example Guiding Channels inside an Enclosed Chamber

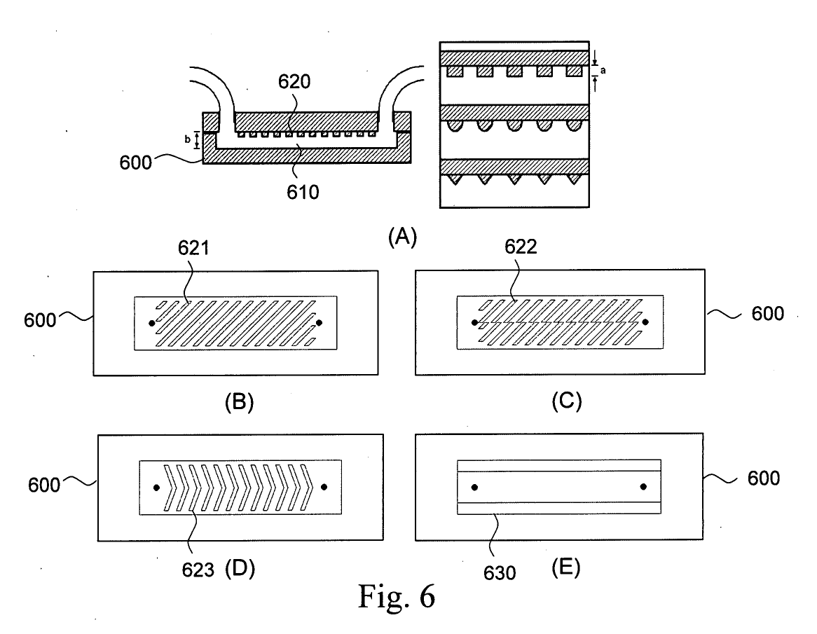

FIG. 6 illustrates some of the possible variations of the fluidic guiding patterns on the cover plate within the enclosed chamber 610 of an example EMC device 600. FIG. 6A shows the cross-sectional view of an example EMC device 600 with fluidic guiding patterns 620 on the inner surface of the cover plate (left). It also shows the dimensions and variations of the cross section of the fluidic guiding patterns (right). FIGS. 6B, 6C, 6D and 6E illustrate some of the ways of patterning the inner surface of the chamber in an EMC device. The top view of the EMC device is used.

In some implementations, one or more arrays of protrusions or indentations 620 are made on the inner surface of the cover plate. The grooves or ridges formed can be parallel at a specific angle to an edge of the chamber or be randomly positioned. The patterns are made for the purpose of changing flow direction inside the enclosed chamber. Because the patterns provide resistance or disturbance to the fluid flow in the enclosed chamber, spin or turbulence may be generated in the flow field and create additional mixing of the fluid inside the chamber. FIG. 6A shows the vertical dimensions of the guiding patterns. The vertical dimension of the enclosed chamber is denoted as “b” and the vertical dimension of the guiding patterns is denoted as “a”. The relative height of the pattern to the chamber “a/b” can be optimized to make most effective flow direction change. A suitable dimension for “a” or “b” can be found by a computational simulation of the flow field, or by other computation based on the physics of fluid flow. In some implementations, the ratio between the vertical dimensions of the pattern and the enclosed chamber is between 0.1 and 0.5. In some implementations the vertical dimensions of the protrusion or indentation is in the range of 0.1 mm to 1 centimeter.

The right of FIG. 6A shows some variations of the cross sectional shape of some protruding patterns. The cross-sectional shape can be practically of any shape, but some shapes may be preferable due to the ease of manufacturing or effectiveness of guiding the flow. In some implementations, the cross-sectional shape of the pattern can be rectangular, semi-circular, or rectangular. The lateral size and spacing of each of the indentation or protrusions can also be adjusted to achieve desired flow pattern. In some implementations, indentations of the same or corresponding shape, size and spacing can be used in place of protrusions.

FIG. 6B, 6C, 6D and 6E illustrate some of the ways of patterning the inner surface of the chamber in an EMC device. FIG. 6B shows an array of slanted linear pattern 621 comprising parallel ridges or grooves at an angle to the longitudinal edge of the enclosed chamber. This type of patterns can add a rotating momentum to the fluid flow in the chamber. The resulted rotating vortex enhances the mixing of different portions in the fluid flow. Because mixing strengthens the mass transport towards the surface of the blotting membrane, the surface-molecular interaction can be significantly accelerated.

FIG. 6C shows multiple arrays of similarly slanted linear patterns 622. Increasing the number of arrays can result in multiple rotating vortices in parallel in the enclosed chamber. In some implementations, the different arrays are placed side by side, with the ridges slightly shifted from each other such as that shown in FIG. 6C.

FIG. 6D shows another configuration of having multiple arrays of slanted linear patterns 623. In this case, the pattern can also generate rotating vortices in parallel but the flow field is different compared to the pattern shown in FIG. 6C. In some implementations, the parallel ridges or grooves in FIG. 6D can be at different angles to an edge of the enclose chamber. In some implementations, the ridges or grooves of different arrays can be connected to each other and form a continuous ridge or groove with kinks in them. In some implementations, the ridge or groves of the patterns of different arrays are parallel but are shifted slightly against each other.

For the patterns shown in FIGS. 6B, 6C, and 6D, there are a few parameters that can be optimized for better performance: (1) angles of the ridges or grooves against an edge of the enclosed chamber; (2) widths of the ridges or grooves compared to the width of the enclosed chamber; (3) spacing between the ridges or grooves in the pattern; and (4) width and number of each arrays within the pattern. In some implementations, the widths of the ridges or grooves are in the range of 0.1 mm to the lateral dimensions of a customarily sized membrane.

FIG. 6E shows a particular pattern of two longitudinal strips 630 along the longitudinal sides of the enclosed chamber. In some implementations, this pattern is placed on the inner surface of the cover plate such that the enclosed chamber created by the cover plate and the support plate is narrower closer to the cover plate and wider closer to the support plate. In some implementations, the cover plate is shaped with a hollowed area in the middle that is narrower than the hollowed area in the support plate, and when the two plates are coupled together they created an enclosed chamber that is narrower closer to the cover plate and wider closer to the support plate. In both cases, the inlet and outlet of the enclosed chamber should be placed in the cover plate at the two longitudinal ends of the enclosed chamber, such that the fluid flow has a greater speed closer to the support plate as compared to the cover plate.

In some implementations, the inner surface of the chamber in an EMC device can be chemically modified for the purpose of guiding fluid flow. The modification can include changing the properties of the surface (for example, by oxidation) or coating the surface with chemical substances (for example, by adsorption of surfactants or by deposition of polymers). These methods influence the hydrophilicity of the surface so that fluid flow can be sped up or slowed down in some portions of or the entire chamber.

### Example EMC Device with Multiple Enclosed Chambers and Assisting Device

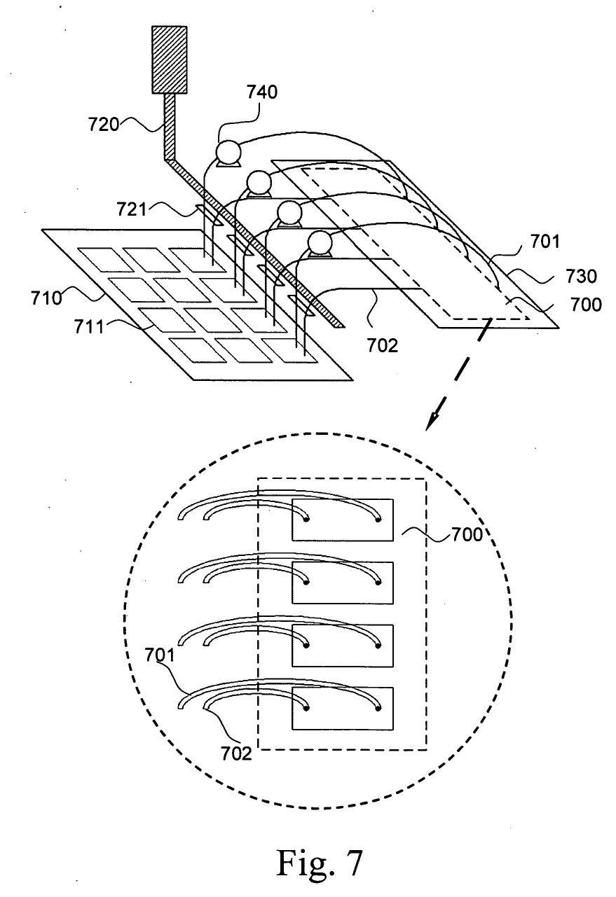

FIG. 7 illustrates the assembly of multiple EMC devices 700 or an EMC device 700 with multiple enclosed chambers and corresponding assisting devices to carry out several biological assays in parallel.

In some implementations, an EMC device can have a single enclosed chamber with one or more pairs of inlet and outlet. In some implementations, a single device may contain multiple enclosed chambers with respective one or more pairs of inlets and outlets. In the case of multiple enclosed chambers in a single EMC device, the cover plate of the EMC device may be a single piece that can be coupled to a single support plate, and forming multiple enclosed chambers simultaneously. In some implementations, the cover plate of each of multiple enclosed chambers in an EMC device may be separate pieces that can be coupled to a single support plate separately and forming the multiple enclosed chambers separately. In some implementations, the cover plate of each of the multiple chambers in an EMC device may be separate pieces and are attached to a single support plate on one edge, and can be coupled to the single support plate separately when in use. In some implementations, the cover plates of an EMC device with multiple enclosed chambers can be reopened and resealed for reuse. In some implementations, one or more features described above with respect to an example single chamber EMC device are still applicable to the multi-chamber EMC device, particularly those with respect to features of the enclosed chamber, the inlets and outlets, and the materials.

In some implementations, a multi-chamber EMC device 700 or multiple single-chamber EMC devices 700 can be assembled with an assisting device to carry out several assays simultaneously. In some implementations, an assisting device can comprise a first support platform 710, a second support platform 720, a third support platform 730 and one or more pumping devices 740 coupled to the one or more enclosed chambers of one or more EMC devices 700. In some implementations, one or more features described above with respect to an example assisting device for a single chamber EMC device (including features for individual components of the assisting device) are still applicable to the assisting device of a multi-chamber EMC device or multiple single-chamber EMC devices.

In some implementations, the first support platform 710 is capable of carrying an array of one or more containers 711 for liquid-phase target solutions. The second support platform 720 is capable of carrying multiple pairs of inlet and outlet tubes 701 and 702, each of which are connected to the pairs of inlet and outlet of the enclosed chambers in the one or more EMC devices 700.

The first and the second platforms are capable of specific relative transverse and vertical movements such that the one or more pairs of inlet and outlet tubes 701 and 702 carried by the second support platform 720 can be submerged in and taken out of each of the containers 711 for liquid-phase target solutions carried by the first support platform 710. In some implementations, the first and/or the second support platform 710 and 720 are capable of individualized movements for each of the one or more single-chamber EMC devices or each of the one or more enclosed chambers in a multi-chamber EMC device. In some implementations, a programmable unit is operably coupled to each of the first support platform and the second support platform, and is capable of controlling and automating the movement of the support platforms for each of the one or more chambers in the one or more EMC devices.

In some implementations, one or more pumping devices 740 are capable of being coupled to the one or more chambers in the one or more EMC devices. The coupling is through one or more pairs of inlet and outlet tubes connected to the one or more enclosed chambers of the one or more EMC devices. In some implementations, the pumping device of the assisting device may be a group of individually functioning pumping devices or a single pumping device having multiple outputs. In some implementations, the pumping device is capable of reversing the pumping directions, either individually or as a group.

In some implementations, a third support platform 730 can be used for carrying the one or more single-chamber EMC devices or multi-chamber EMC devices. The third support platform 730 is capable of orienting the one or more EMC devices together or separately such that the outlet is higher than the inlet of each of the one or more chambers in the EMC device when the first target or each of the one or more second targets is to be continually pumped into the chamber, and orienting the EMC device such that the outlet is lower than the inlet when the first target and each of the one or more second targets are being emptied out of each of the one or more chambers.

In some implementations, the pumping device 740 is capable of reversing pumping direction and the third support platform 730 does not need to move. In such implementations, the EMC devices can be fixed in orientation and the outlet 702 is higher than the inlet 701 of each enclosed chambers in the one or more EMC devices 700 when the first target or each of the successive targets is to be continually pumped into each of the enclosed chambers. When emptying each of the enclosed chambers in preparation of the injection of the next target, the pumping direction is reversed and the roles of the inlet and outlet are temporarily reversed and the liquid inside the chamber is emptied out via the inlet of the chamber.

In some implementations, a feature 721 is applied to affix the inlet and outlet tubes from one or more enclosed chambers of one or more EMC devices. The purpose of this feature is to keep the positions of the tubes and their separation distances. In most cases, the ends of inlet and outlet tubes are close enough to keep them submerging in the same container for a liquid-phase target solution, at the same time, the separation between the ends of the inlet and outlet tubes should be sufficiently large to ensure that fresh target solution is circulated in the chambers of the EMC devices.

In some implementations, the clamping feature of the cover plate as described above for the example EMC device with a single enclosed chamber can be repeated for each of the enclosed chamber in a multi-chamber EMC device.

In some implementations, the sealing mechanism as described above for an example EMC device with a single enclosed chamber can be repeated for each of the enclosed chambers in a multi-chamber EMC device.

FIG. 7 illustrates the concept that multiple EMC devices or a multi-chamber EMC device can be used to carry out multiple assays simultaneously. In some implementations, with a suitable assisting device, the multiple assays can be carried out automatically.

### Example Procedures for Using an EMC Device in Running Assays

In some implementations, an EMC device can be used to run biological assays such as Western blotting, Northern blotting, Southern blotting, or dot blotting. As an example, a typical Western blotting assay includes three major steps: (1) performing sodium dodecyl sulfate-polyacrylamide gel electrophoresis (SDS-PAGE) of a sample composed of proteins (these are targets to be identified or quantified); (2) transferring these targets to a blotting membrane thus they become immobilized targets; and (3) generating and observing detectable signals through surface-molecular interactions taking place on the blotting membrane between the immobilized targets and one or more mobilized targets in each of a liquid-phase one or more successive liquid-phase target solutions.

In most cases, the first liquid-phase target solution used is an antibody that can specifically bind to one of the compositions of the immobilized targets transferred to the membrane (if applied to a blotting membrane containing only the immobilized target to be detected). In some implementations, at least one of one or more successive liquid-phase targets is capable of generating calorimetric or fluorescent or other type of detectable signals for the purpose of identification or quantification of the compositions of the immobilized target initially transferred to the membrane. An EMC device is designed to carry out surface-molecular interactions with high efficiency, so that it is capable of achieving the third major step with acceleration and automation.

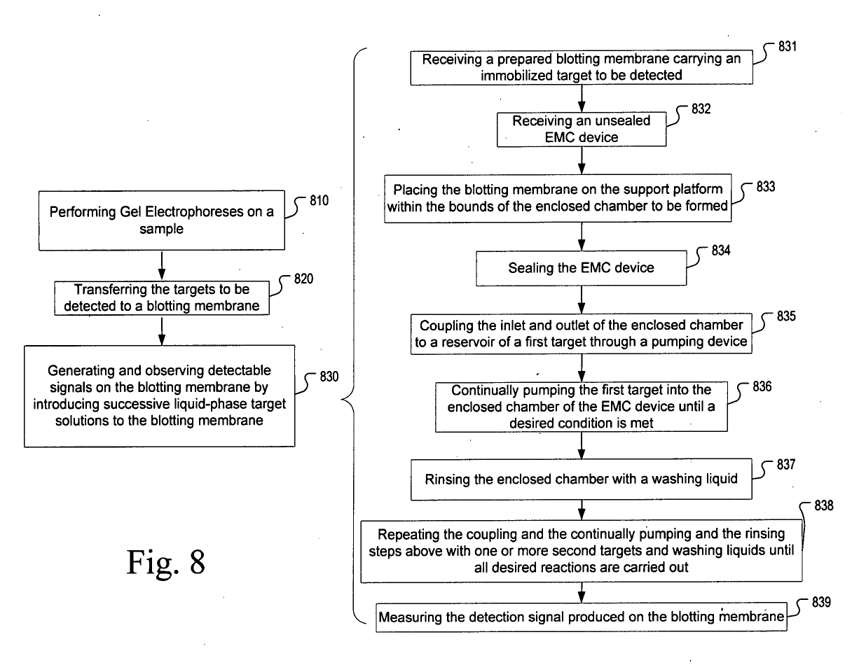

FIG. 8 is a flow diagram of an example process for carrying out a blotting assay using an EMC device. FIG. 8 describes a typical procedure of using an EMC device to achieve Western blotting as an illustration of how to apply the EMC device. The left side of FIG. 8 shows the three major steps (810, 820 and 830) in a typical Western blotting, and the right side of FIG. 8 shows the some of the typical procedures of realizing the third major step (830) of a typical Western blotting assay using an EMC device (831-839).

A typical process can include some or all of the following steps:

(1) receiving a customarily sized blotting membrane already stained with immobilized targets to be identified or quantified (Step 831 in FIG. 8).

(2) receiving an unsealed EMC device as those described above (Step 832 in FIG. 8).

(3) placing the customarily sized blotting membrane on the support plate within the bounds of the enclosed chamber to be formed by the cover plate, the support plate, and the sealing mechanism of the EMC device (Step 833 in FIG. 8).

(4) sealing the cover plate and the support plate together through the sealing mechanism to form an enclosed chamber such that the blotting membrane is sealed inside the enclosed chamber (Step 834 in FIG. 8).

(5) coupling one or more inlet tubes to the one or more inlets of the enclosed chamber containing the blotting membrane and coupling one or more outlet tubes to the one or more outlets of the enclosed chamber containing the blotting membrane. (This step is optional if the EMC device already come with the tubes attached).

(6) coupling one or more pumping devices to the one or more inlet or outlet of the enclosed chamber containing the blotting membrane.

(7) coupling the one or more inlet and outlet tubes to a reservoir of a liquid-phase first target and continually pumping the liquid-phase first target into the one or more inlets, through the enclosed chamber, and out of the one or more outlets of the enclosed chamber containing the blotting membrane until a desired condition is met (Steps 835 and 836 in FIG. 8).

(8) orienting the enclosed membrane-clamping device such that the outlet is higher than the inlet of the enclosed chamber when each of the successive liquid-phase target solutions is to be continually pumped into the enclosed chamber.

(9) orienting the enclosed membrane-clamping device such that the outlet is lower than the inlet when each of the successive liquid-phase target solutions is being emptied out of the enclosed chamber.

(10) after sufficient reaction time or other desired condition is met, rinsing the enclosed chamber which comprises the following steps: first expelling all fluids out of the enclosed chamber, and then injecting a washing liquid into the enclosed chamber, and then expelling the washing liquid from the enclosed chamber (Step 837 in FIG. 8).

(11) after rinsing, coupling the one or more inlet and outlet tubes to a reservoir of a liquid-phase second target and continually pumping the liquid-phase second target into the one or more inlets, through the enclosed chamber, and out of the one or more outlets of the enclosed chamber containing the blotting membrane until a desired condition is met.

(12) the steps of rinsing the enclosed chamber, coupling the one or more inlets and outlets of the enclosed chamber to a reservoir of a second target, and continually pumping the second target into the enclosed chamber can be repeated with different desired second targets until reactions with all desired second targets are carried out and the generation of detection signal on the blotting membrane is completed (Steps 838 in FIG. 8).

(13) After the generation of detection signals is completed, emptying the chamber containing the blotting membrane.

(14) measuring the detection signals on the blotting membrane (Steps 839 in FIG. 8). In some implementations, the detection signals may be measured by directly observing the blotting membrane through the cover plate of the EMC device. In some implementations, the EMC device may be opened to release the blotting membrane, and the measurement is done by observing the blotting membrane directly or with additional processing. In some implementations, if the generation of observable detection signals needs the presence of an additional reactive target, such as in a traditional chemiluminescence method, the chamber may be filled with the additional reactive target while measuring the signals.

The procedures can also be done using an assisting device as described above, which can further include one or more of the following steps:

(1) placing the liquid-phase first target and one or more desired liquid-phase second targets in different containers on a first support platform;

(2) affixing the pair of inlet and outlet tubes to a second support platform, wherein the first and second support platforms are capable of specific transverse and vertical movements such that the tips of the pair of inlet and outlet tubing can be submerged and taken out of each of the first and one or more desired second targets carried by the first support platform.

(3) placing the EMC device on the third support platform.

(4) coupling the inlet and outlet of the enclosed chamber to the pumping device through the inlet and outlet tubes.

(5) adding liquid-phase first target and one or more liquid-phase second targets to containers carried by the first support platform.

(6) Setting a programmable unit of the assisting device to an “interaction mode” to continually pump the first or second target into the chamber through the inlet and outlet until a desired condition is met. The programmable unit is operably coupled to the first, second and third support platforms and moves them to submerge and take out the ends of the inlet and outlet tubes from each of the containers of liquid-phase targets at specific times or when a desired condition is satisfied. The desired condition is usually met when the equilibrium of a surface-molecular interaction between the targets attached to the blotting membrane and the targets in the liquid-phase solutions. Specific time for starting and ending a reaction can also be determined based on experience of the user.

The assisting device can also be turned to a “rinsing mode” where the programmable unit coupled to the first, second, and third platform can carry out the functions of expelling the liquid-phase target solution out of the chamber, and pumping a washing fluid into the chamber and then emptying the enclosed chamber of all liquids.

The assisting device can also repeat the “interaction mode” and “rinsing mode” for one or more other liquid-phase second targets until reactions with all the desired second targets are carried out and the desired identification or quantification is completed.

In some implementations, the assisting device is computer controlled and all of the steps can be automated. In some implementations, the timing, duration, parameters of each step may be individually programmed to satisfy different user's requirements.

In some implementations, if any of the targets involved is hazardous, such as radioactive, the assisting device together with the EMC device may be enclosed in a protective container, such as a metal box to protect the safety of users.

In some implementations, an EMC device together with the assisting device may be applied to the biological assays for gas-phase target solutions, which means one or more targets are dissolved in gas and introduced into the chamber for surface-molecular interactions taking place on the surface of the blotting membrane.

While this specification contains many specifics, these should not be construed as limitations on the scope of what is being claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understand as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described components and systems can generally be integrated together in a single product or packaged into multiple products.

Thus, particular embodiments have been described. Other embodiments are within the scope of the following claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such may vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

In describing and claiming the disclosed subject matter, the following terminology will be used in accordance with the definitions set forth below.

In accordance with the present disclosure there may be employed conventional biochemistry, microbiology, and recombinant DNA techniques within the skill of the art. Such techniques are explained fully in the literature, such as “Molecular Cloning: A Laboratory Manual” (J. Sambrook & D. W. Russell ed. 2001); “DNA Cloning: A Practical Approach” (D. N. Glover ed. 1995); “Oligonucleotide Synthesis” (M. J. Gait ed. 1984); “Nucleic Acid Hybridization” (B. D. Hames & S. J. Higgins ed. 1985); “Transcription and Translation” (B. D. Hames & S. J. Higgins ed. 1984); “Immobilized Cells and Enzymes” (J. Woodward ed. 1985); “A Practical Guide to Molecular Cloning” (B. Perbal ed. 1988); “Antibodies: A Laboratory Manual” (E. Harlow & D. P. Lane ed. 1988), each of which is incorporated herein by reference.

As used herein, the term “interaction” can include chemical interactions and biological interactions. The interactions include, but are not limited to, chemical bonding including covalent and non-covalent bonding, biochemical interaction, physical interaction, chelation interaction, hydrophobic interactions, hydrophilic interactions, charge-charge interactions, π-stacking interactions, combinations thereof, and otherwise associated with one another among one or more functional groups located on the molecules involved in the interaction.

As used herein, the term “target” is intended to encompass molecules participating in the interactions taking place in an MEC device, including chemical targets and biological targets, deoxyribonucleic acids (DNA), ribonucleic acids (RNA), nucleotides, oligonucleotides, nucleosides, polynucleotides, proteins, peptides, antibodies, antigens, ligands, receptors, protein complexes, and combinations thereof. In particular, a chemical target includes, but is not limited to, organic compounds, inorganic compounds, surfactants, polymers, pathogens, toxins, combinations thereof, and the like. A biological target includes, but is not limited to, bioactive molecules such as deoxyribonucleic acids (DNA), ribonucleic acids (RNA), nucleotides, oligonucleotides, nucleosides, polynucleotides, proteins, peptides, antibodies, antigens, ligands, receptors, protein complexes, combinations thereof, and the like, and naturally occurring substances such as micelles, vesicles, eukaryotic cells, prokaryotic cells, microorganisms such as viruses, bacteria, protozoa, archaea, fungi, algae, spores, combinations thereof, and the like.

The term “sample” is used herein to refer to an object containing a target of interest which is to be detected on a blotting membrane in an MEC device. The sample mentioned in the present disclosure mainly refers to fluidic samples in gas phase or liquid phase including suspensions, in most cases aqueous solutions. A sample may contain components besides the target of interest.

The term “reagent” is used herein to refer to an object containing a target which is used to facilitate the process of the target of interest immobilized on a blotting membrane in an MEC device. The reagent mentioned in the present disclosure mainly refers to fluidic reagents in gas phase or liquid phase including suspensions, in most cases aqueous solutions.

As used herein, the term “biological assay” refers to all assays used in biological sciences and technologies, including protein interaction assays such as immunoassays, nucleic acid hybridization assays, protein-nucleic acid interaction assays, membrane assays, heterogeneous phase assays such as enzyme-linked immunosorbent assay (ELISA) and western blot, homogeneous phase assays such as measurement of optical density (OD) value, reaction assays such as immunoprecipitation (IP), mechanical force assays such as centrifuge and sedimentation, chromatographic assays such as affinity chromatography, combinations thereof, and the like. The assays are used for the purpose of manipulating targets of interest in biological samples, including target detection, quantification, extraction, and the like.

The term “fluidic channel network” used herein can refer to an interconnected system of one or more fluidic channels between inlets and outlets. The dimensions of the fluidic channels may vary from sub-micrometers to millimeters. The fluidic channels can be of any structure, such as rectangular, circular, elliptic, and the like, and the cross-section can be rectangular or round. Within the microfluidic channels, the surfaces can be chemically and/or physically modified for the purpose of enhancing the contact efficiency between mobile and immobilized targets, such as coating the surfaces to adjust the surface adsorption of targets, creating patterns on the surfaces to generate turbulences in fluidic flows.

As used herein, the term “antibody” includes, but are not limited to, monoclonal antibodies, multispecific antibodies, human antibodies, humanized antibodies, camelised antibodies, chimeric antibodies, single-chain Fvs (scFv), single chain antibodies, Fab fragments, disulfide-linked Fvs (dsFv), and anti-idiotypic (anti-Id) antibodies (e.g., anti-Id antibodies to antibodies of the disclosure), and epitope-binding fragments of any of the above. In particular, antibodies include immunoglobulin molecules and immunologically active fragments of immunoglobulin molecules (e.g., molecules that contain an antigen binding site). Immunoglobulin molecules can be of any type (e.g., IgG, IgE, IgM, IgD, IgA and IgY), class (e.g., IgG1, IgG2, IgG3, IgG4, IgA1 and IgA2), or subclass. The antibodies may be from any animal origin including birds and mammals (e.g., human, murine, donkey, sheep, rabbit, goat, guinea pig, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. The antibodies may be monospecific, bispecific, trispecific, or of greater multispecificity.

As used herein, “antigen” describes a compound, a composition, or a substance that can stimulate the production of antibodies or a T-cell response in a host.

What is claimed:

1. An enclosed membrane-clamping device, comprising:
a cover plate;
a support plate;
a sealing mechanism, wherein
the cover plate and the support plate each has an inner surface facing each other; and wherein
the sealing mechanism is located on either the cover plate, the support plate, or both plates, or is a standalone part capable of attaching to both plates; and wherein
the cover plate, the support plate and the sealing mechanism are shaped such that their inner surfaces form one or more enclosed chambers when the cover plate and the support plate are coupled together by the sealing mechanism; and wherein
lateral dimensions of the inner surface area on the support plate within each of the one or more enclosed chambers are sufficient to accommodate a customarily sized blotting membrane for purposes of running biological assays; and wherein
vertical dimension of each of the one or more enclosed chambers are in the range of 0.1 millimeter to 1 centimeter;
an inlet for the inflow of a target fluid into each of the one or more enclosed chambers; and
an outlet for the outflow of the target fluid from each of the one or more enclosed chambers.

2. The enclosed membrane-clamping device of claim 1, wherein
the sealing mechanism is a double-sided adhesive tape.

3. The enclosed membrane-clamping device of claim 1, wherein
the cover plate, the support plate, and the sealing mechanism are shaped or contain additional parts such that one or both ends of a customarily sized blotting membrane placed within the bounds of one of the enclosed chambers can be clamped to the inner surface of the support plate by the cover plate or the sealing mechanism when the cover plate and the support plate are coupled together by the sealing mechanism.

4. The enclosed membrane-clamping device of claim 1, wherein
the inner surface of the cover plate within the bounds of at least one of the one or more enclosed chambers is patterned with one or more protrusions or indentations on the inner surface of the cover plate such that additional mixing of the target fluid is produced.

5. The enclosed membrane-clamping device of claim 4, wherein
the one or more protrusions or indentations are formed by one or more arrays of protrusions or indentations, wherein
each of the one or more protrusions or indentations has a corresponding vertical dimension;
the ratio between vertical dimension of each of the one or more protrusions or indentations and the vertical dimension of the one or more enclosed chambers are in the range of 0.1-0.5; and wherein
the lateral dimensions of each of the protrusions or indentations are in the range of 0.1 millimeter to the lateral dimension of the at least one of the one or more enclosed chambers.

6. Then enclosed membrane-clamping device of claim 5, wherein
one of the one or more arrays of protrusions or indentations comprises parallel grooves or ridges running at a specific angle to an edge of the at least one of the one or more enclosed chambers.

7. The enclosed membrane-clamping device of claim 1, wherein
the inlet and the outlet of one of the one or more enclosed chamber are located longitudinally on the cover plate at two opposite ends of the enclosed chamber; and wherein
the one of the one or more enclosed chamber is narrower closer to the cover plate and wider closer to the support plate in the direction across the enclosed chamber.

8. The enclosed membrane-clamping device of claim 1, wherein
the inlet of one of the one or more enclosed chambers are connected to channel networks within the enclosed chamber that distributes the target fluid to desired locations along the direction across the enclosed chamber.

9. A device for using an enclosed membrane-clamping device having one or more enclosed chambers and corresponding pairs of inlet and outlet in running a biological assay, comprising
a first support platform capable of carrying one or more containers for target fluids;
a second support platform capable of carrying one or more pairs of inlet and outlet tubes each of which is coupled to an enclosed chamber of the enclosed membrane-clamping device via its pair of inlet and outlet, and wherein
the first and the second platforms are capable of specific relative transverse and vertical movements such that the one or more pairs of inlet and outlet tubes carried by the second support platform can be submerged in and taken out of each of the one or more containers for target fluids carried by the first support platform;
a pumping device capable of being coupled to the one or more enclosed chambers of the enclosed membrane-clamping device and driving the movement of target fluids into the inlets, through the one or more enclosed chambers, and out of the outlets of the enclosed membrane-clamping device.

10. The device of claim 9, further comprising:
a programmable unit operably coupled to the first and/or the second support platform, and capable of controlling the timing and duration of relative movements of the first and second support platform.

11. The device of claim 10, further comprising
a third support platform for carrying the enclosed membrane-clamping device, and wherein
the programmable unit is operably coupled to the third support platform and
the third platform is capable of orienting the enclosed membrane-clamping device such that the outlet is higher than the inlet of the enclosed chamber when a target fluid is being continually pumped into the enclosed chamber, and that the outlet is lower than the inlet when the target fluid is being emptied out of the enclosed chamber.

12. The device of claim 10, wherein:
the programmable unit is operably coupled to the pumping device and can reverse the pumping direction of the pumping device at specified or programmed times.

13. A method of using an enclosed membrane-clamping device in running a biological assay, comprising:
receiving an enclosed membrane-clamping device having
a cover plate;
a support plate;
a sealing mechanism, wherein
the cover plate and the support plate each has an inner surface facing each other; and wherein
the sealing mechanism is located on either the cover plate, the support plate, or both plates, or is a standalone part capable of attaching to both plates; and wherein
the cover plate, the support plate and the sealing mechanism are shaped such that their inner surfaces form one or more enclosed chambers when the cover plate and the support plate are coupled together by the sealing mechanism; and wherein
lateral dimensions of the inner surface area on the support plate within each of the one or more enclosed chambers are sufficient to accommodate a customarily sized blotting membrane for purposes of running biological assays; and wherein
vertical dimension of each of the one or more enclosed chambers are in the range of 0.1 millimeter to 1 centimeter;
an inlet for the inflow of a target fluid into each of the one or more enclosed chambers; and
an outlet for the outflow of the target fluid from each of the one or more enclosed chambers.
placing a customarily-sized blotting membrane on the support plate and within the bounds of one of the enclosed chambers to be formed;
coupling the cover plate and the support plate together with the sealing mechanism such that the blotting membrane is sealed inside one of the enclosed chambers formed;
coupling the inlet and outlet of the enclosed chamber containing the blotting membrane to a reservoir of a first target via a pumping device;
continually pumping the first target into the enclosed chamber through the inlet and out of the enclosed chamber through the outlet of said enclosed chamber until a desired condition is met.

14. The method of claim 13, further comprising:
rinsing the enclosed chamber which comprises the following steps:
first expelling all fluids out of the enclosed chamber, and then
injecting a washing liquid into the enclosed chamber, and then
expelling the washing liquid from the enclosed chamber.

15. The method of claim 14, further comprising:
coupling the inlet and outlet of the enclosed chamber containing the blotting membrane to a reservoir of a second target via a pumping device;
continually pumping the second target into the enclosed chamber through the inlet and out of the enclosed chamber through the outlet of said enclosed chamber until a desired condition is met.

16. The method of claim 15, wherein:
the steps of rinsing the enclosed chamber, coupling the inlet and outlet of the enclosed chamber to a reservoir of a second target, and continually pumping the second target into the enclosed chamber can be repeated with different desired sample fluids serving as the washing liquid and the second target.

17. The method of claim 16, wherein
the coupling the inlet and outlet of the enclosed chamber containing the blotting membrane to a reservoir of a first or second target via a pumping device is through a pair of inlet and outlet tubes; and
the method further comprises:
placing the first target and one or more desired second target in different containers on a first support platform;
affixing the pair of inlet and outlet tubes to a second support platform, wherein the first and second support platforms are capable of specific transverse and vertical movements such that the tips of the pair of inlet and outlet tubing can be submerged and taken out of each of the first and one or more desired second target carried by the first support platform.

18. The method of claim 17, further comprising
moving the first support platform, or the second support platform, or both, to submerge the tips of the pair of inlet and outlet tubes into the container containing the desired first target or second target; and
moving the first support platform, or the second support platform or both to separate the tips of the pair of inlet and outlet tubes from the container containing the desired first target or second target.

19. The method of claim 18, further comprising
affixing the enclosed-membrane-clamping device to a third support platform;
orienting the enclosed membrane-clamping device such that the outlet is higher than the inlet of the enclosed chamber when the first target or each of the one or more second target is to be continually pumped into the enclosed chamber, and
orienting the enclosed membrane-clamping device such that the outlet is lower than the inlet when the first target and each of the one or more second target is being emptied out of the enclosed chamber.

20. The method of claim 19, wherein
the steps of moving, pumping, rinsing, and orienting are carried out automatically via a programmable unit coupled to the first support platform, the second support platform, and the third support platform, and at specified or programmable times and for specified or programmable durations.
